(19) United States
(12) Patent Application Publication
(10) Pub. No.: US 2005/0137708 A1
(43) Pub. Date: Jun. 23, 2005

(54) Device and method of arthroscopic knee joint resurfacing

(76) Inventor: Ron Clark, Valparaiso, IN (US)

Correspondence Address: MARK S. MORRISON, 1508 Springhill Road, Warsaw, IN, 46580, US

(21) Appl. No.: 11/020,398

(22) Filed: Dec. 22, 2004

Related U.S. Application Data
(60) Provisional application No. 60/532,537, filed on Dec. 23, 2003.

(51) Int. Cl. A61F 2/38

(52) U.S. Cl. 623/20.15; 623/20.3; 623/20.32; 623/20.35

(57) ABSTRACT

A knee joint resurfacing implant is disclosed. The knee joint resurfacing implant may include femoral implant and tibial implant components. The femoral implant components may be attached to the femur using screws or other fixation devices. The femoral implant component may be configured to share loads between cortical and cancellous bone material. The tibial implant components are formed in modular portions which may be assembled within the knee joint and may be free-floating or fixed to the tibial surface. The implant components are of such a size and configuration that the components may be placed into the knee joint through an external skin incisions that measure 3 cm or less. Accordingly, the size of skin incisions required for implantation may be reduced when compared to incisions which are now commonly used.

### CROSS-REFERENCE TO RELATED APPLICATIONS

This application claims-priority from provisional patent application Ser. No 60/532,537, filed with the United States Patent and Trademark office on Dec. 23, 2003. The entire teachings of the said application are incorporated herein by reference.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

The present disclosure relates generally to knee replacement implants, and more particularly, but not necessarily entirely, to arthroscopic knee replacement implants for the femur and tibia.

It is common to provide implants to resurface worn articular surfaces of knees. The sizes of incisions required to insert the implants are often relatively large. Moreover, the prior art is characterized by several other disadvantages that are addressed by the present disclosure. The invention of the present disclosure minimizes, and in some aspects eliminates, numerous failures of the prior art, and other problems, by utilizing the devices described herein.

### BRIEF SUMMARY OF THE INVENTION

A knee joint resurfacing implant including femoral and tibial implant components is disclosed. The femoral implant component includes an apex and supports on the proximal surface which are configured to share loads between cortical and cancellous bone material when the femoral implant component is implanted on the distal end of a prepared femur. The femoral implant may be attached to the femur using screws or other rigid members such as a peg, pin, or stem that traverses the external articulating surface and has a terminal surface that uniformly matches the external articulating surface of the implant. The tibial implant component of the knee replacement implant has a semicircular circumference, a concave superior surface, a planar inferior surface, and is divided into medial and lateral halves by a joint running longitudinally through the implant. The tibial implant component may be assembled within the knee joint. Accordingly, the femoral and tibial implant components may be configured and sized to be implanted using an arthroscopic surgical techniques and to permit the reduction of the size of skin incisions required for implantation.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a front view of a femoral implant in accordance with the principles of the present disclosure;

FIG. 2 is a side view of the femoral implant of FIG. 1;

FIG. 3 is a cross-sectional view of the femoral implant taken along section A-A of FIG. 2;

FIG. 4 is a top view of a tibial implant;

FIG. 5 is a side view the tibial implant of FIG. 4, as viewed from the lateral side;

FIG. 6 is an end view of the tibial implant of FIG. 4; and

FIG. 7 is an exploded side view of a knee, including a femur, tibia, and patella, in which the femoral and tibial implants are installed.

### DETAILED DESCRIPTION OF THE INVENTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

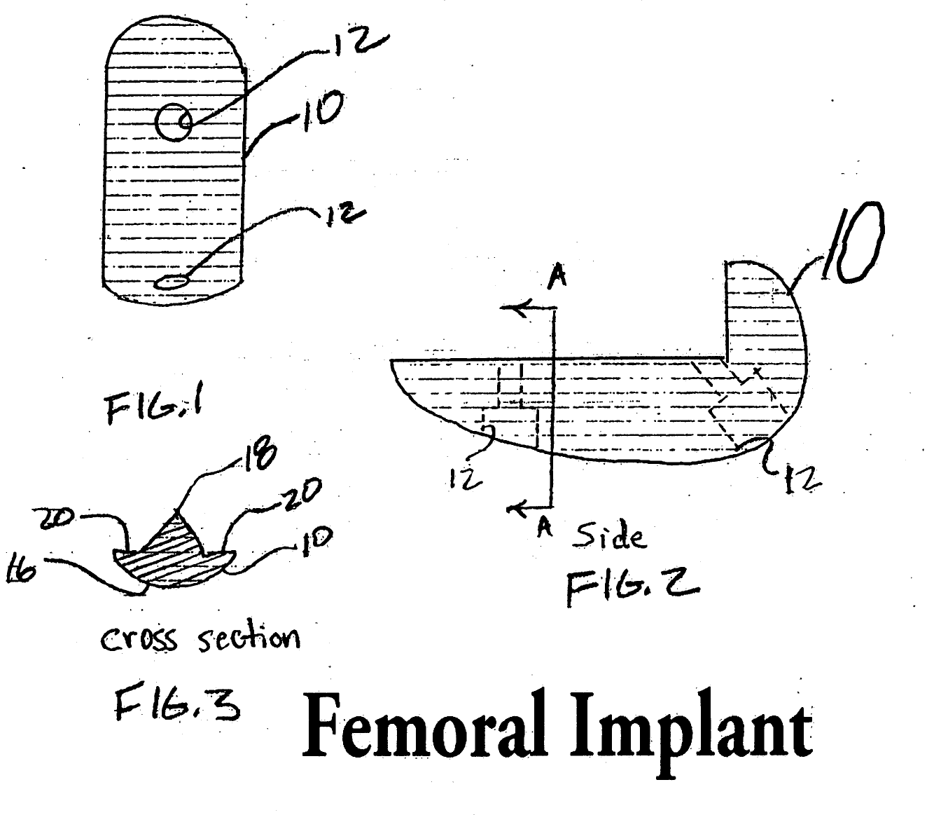

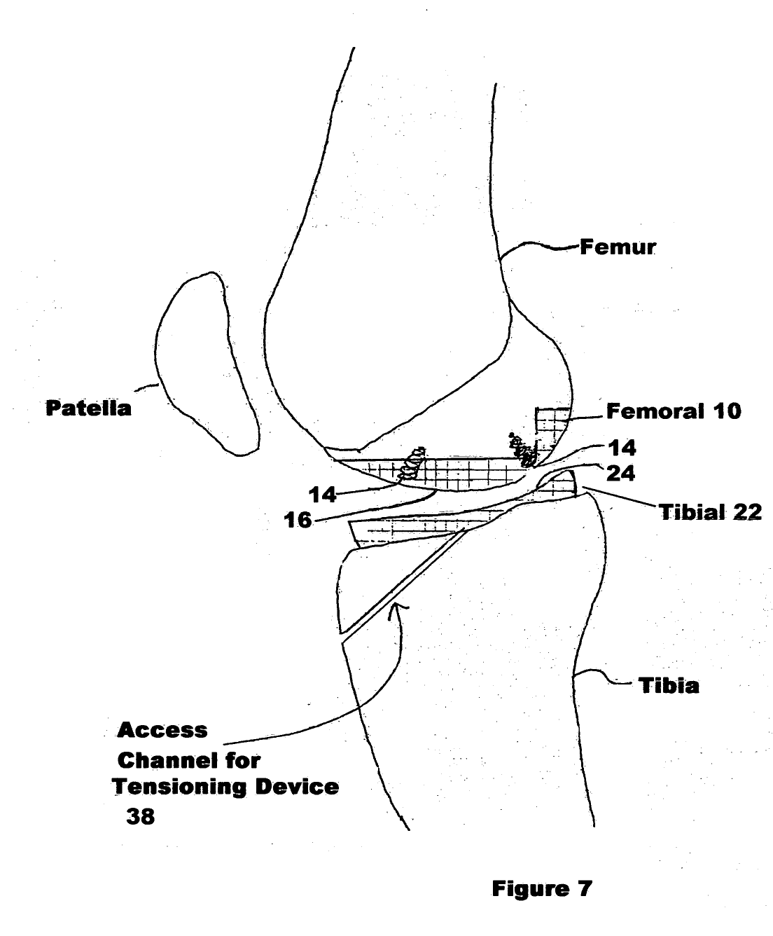

Referring now to FIG. 1, a front view of a femoral implant 10 is shown. The femoral implant 10 may have one or more screw receiving holes 12 for receiving a screw 14 for attaching the femoral implant 10 to the distal end of the femur, as shown most clearly in FIG. 7. As shown in FIG. 3, which shows a cross-sectional view of the femoral implant 10 taken along the line A-A in FIG. 2, the femoral implant 10 may include a curved surface 16 configured for contacting a tibial implant 22 as shown in FIG. 7. The femoral implant 10 may also include an apex portion 18 and supports 20 to engage the prepared distal end of the femur. The apex portion 18 may be configured to be received in the cancellous bone material of the femur, whereas the supports 20 may be configured to contact the cortical bone material of the femur, so that load sharing between the cortical bone and the cancerous bone may be accomplished.

The femoral implant 10 may be used to repair a single condyle, referred to as a unicondylar replacement. Also, two femoral implants 10 may be used in a bi-condylar replacement within the scope of the present disclosure.

The femoral component 10 may be shaped to reproduce the weight bearing articular surface of the knee. The femoral component 10 may be configured to be 15-20 mm in width, for example, with an anatomical assortment of lengths and thicknesses. It will be understood that other widths and dimensions may be used within the scope of the present disclosure. Moreover, it will be understood that the femoral implant 10 may be implanted without resurfacing the entire width of the condyle. Accordingly, the femoral implant 10 may form a narrow rim on the condyle which may cooperate with the remaining portion of the condyle. The femoral component 10 may be made of a high molecular weight polyethylene (HMWPE) or any other suitable material known to those skilled in the art.

The screw receiving holes 12 may be formed of two distinct dimensions, as shown most clearly in FIG. 2, to allow for a cancellous type of screw to pass in order to provide fixation to the femur, with or without polymethylmethacrylate (PMMA) cement.

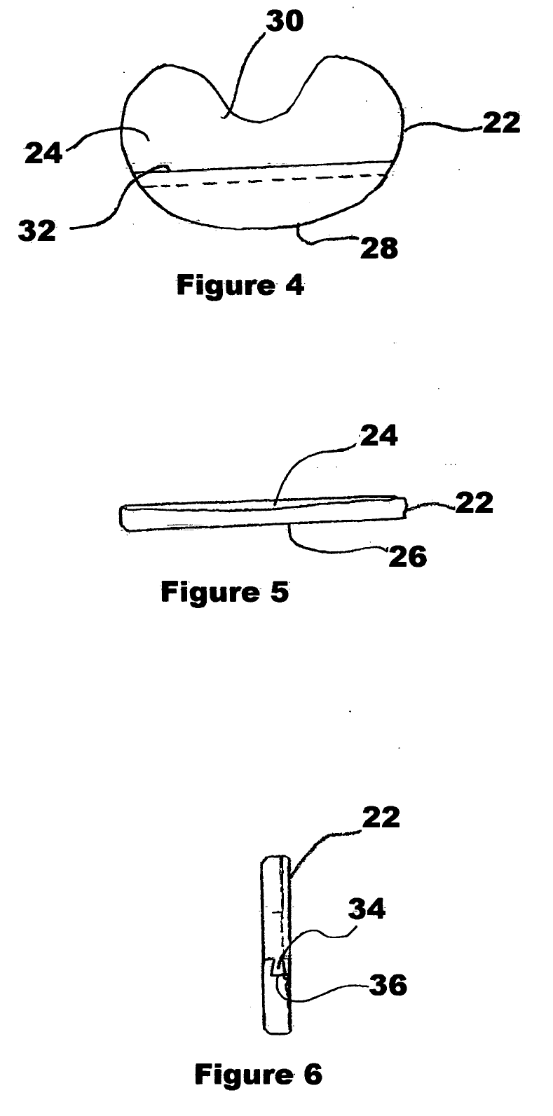

As shown in FIGS. 4-7, the tibial implant component 22 may be formed having a semicircular circumference. The tibial implant component 22 may have a concave superior surface 24 and a planar inferior surface 26. The tibial implant component 22 may be divided into a medial portion 28 and a lateral portion 30 by an implant joint 32. The implant joint 32 may allow the tibial implant component 22 to be inserted into the knee joint in separate pieces through an arthroscopic portal and then assembled within the knee joint at the time of implantation under arthroscopic visualization. It will be understood that the implant joint 32 may be in the form of a dovetailed joint with a projection 34 on the lateral portion 30 and a channel 36 on the medial portion 28, or the projection 34 may be formed on the medial portion 28 and the channel 36 may be on the lateral portion 30. It will also be appreciated that other types of junctions or interlocking geometric interfaces may be used within the scope of the present disclosure. Moreover, the implant joint 32 may be self locking and may include any of a variety of fasteners or attaching devices to prevent the medical portion 28 and the lateral portion 30 from becoming separated.

It will be understood that the tibial implant 22 may be formed of metal, polymer, or any other suitable material known to those skilled in the art.

The tibial implant component 22 may be attached to the tibia to prevent migration of the tibial implant component 22 with respect to the tibia, or the tibial implant component 22 may be free floating on the tibia. The tibial implant component 22 may be cemented in place using an access channel 38 passing obliquely from the anterior proximal tibia into the central aspect of the tibial plateau as illustrated in FIG. 7. The access channel 38 allows cement to be placed on the tibial implant 22 in a manner to avoid unwanted spreading of the cement. Moreover, a strand or other tensioning device may optionally be placed in the access channel 38 and attached to the tibial implant component 22 by any suitable means. The strand may be a suture or any other mechanism known to those skilled in the art. Similarly, any variety of cement known to those skilled in the art may be installed through the access channel 38.

As shown in FIG. 7, which shows an exploded side view of a knee, including the femur, tibia and patella, in which the femoral implant component 10 and tibial implant component 22 are implanted, the convex curved surface 16 of the femoral implant component 10 may be configured to fit in the concave superior surface 24 of the tibial implant component 22. The inferior surface 26 of the tibial implant component 22 as shown in FIG. 5 is configured to mate to the proximal surface of the tibia as shown in FIG. 7, and the superior surface 24 of the tibial implant component 22 is configured to mate and articulate with the distal convex curved surface 16 of the femoral component 10 in a conformational relationship. Thus, the size and shape of the tibial implant 22 and femoral implant 10 and their articulating surfaces may vary to conform to a desired anatomical structure. In one embodiment of the tibial implant 22, the tibial implant 22 may be thicker on the medial side 28 than on the lateral side 30.

Orientation of the mechanical axis of the knee joint, and the depth and position of cuts made to the native surface of the knee prior to implant insertion may be facilitated by the use of a surgical navigation system that may include immediate real time data acquisition and retrieval.

The present disclosure provides surgeons and patients with a reliable and technical method and implants to appropriately resurface the worn articular surface of a single knee compartment. The design of the instruments and implants may allow skin incisions to be less than 25 mm in length. The procedure may be performed arthroscopically with surgical navigational assist.

In the foregoing, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the claims will reflect, inventive aspects lie in less than all features of a single foregoing disclosed embodiment.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

1. An implant to resurface a femoral condyle of the knee joint comprising a femoral body that has a distal, convex surface for articulation with the tibial side of the joint and a proximal surface that has an apex for engagement with the distal end of a prepared femur.

2. The implant of claim 1 wherein the profile of the distal, convex surface closely approximates the radius of curvature of the articulating surface of a native femoral condyle in the sagital plane.

3. The implant of claim 1 wherein fixation of the implant to the femur is provided by one or more screws that are inserted in one or more screw receiving holes that traverse the implant through the femoral body from the distal, convex surface to the proximal surface.

4. The implant of claim 1 wherein fixation is provided by one or more rigid fixation members, such as a screws, pegs, pins or stems, that are inserted in one or more screw receiving holes that traverse the implant through the femoral body from the distal, convex surface to the proximal surface and that traverse the external articulating surface and have a terminal surface profile that uniformly matches the distal, convex articulating surface of the implant such that when fully seated the terminal surface of said fixation members is flush or congruent with the external articulating surface of the implant.

5. The implant of claim 1 wherein the implant is formed from a biologically compatible metal, polymer, biologic, allograft, or xenograft material.

6. The implant of claim 1 wherein fixation of the implant to the femur is augmented by bone cement or adhesive that is be placed between the implant and the femur.

7. The implant of claim 1 wherein the implant is of such a size and configuration that it may be placed into the knee joint through an external skin incision that measures 3 cm or less.

8. The implant of claim 1 wherein the implant may be implanted using an arthroscopic visualization method.

9. An implant to resurface a femoral condyle of the knee joint comprising a femoral body that has a distal, convex surface for articulation with the tibial side of the joint and a proximal surface that has an apex for engagement with cancellous bone of the distal end of a prepared femur and one or more supports for engagement with cortical bone of the distal end of a prepared femur.

10. The implant of claim 9 wherein the profile of the distal, convex surface closely approximates the radius of curvature of the articulating surface of a native femoral condyle in the sagital plane.

11. The implant of claim 9 wherein fixation of the implant to the femur is provided by one or more screws that are inserted in one or more screw receiving holes that traverse the implant through the femoral body from the distal, convex surface to the proximal surface.

12. The implant of claim 9 wherein fixation is provided by one or more rigid fixation members, such as a screws, pegs, pins or stems, that are inserted in one or more screw receiving holes that traverse the implant through the femoral body from the distal, convex surface to the proximal surface and that traverse the external articulating surface and have a terminal surface profile that uniformly matches the distal, convex articulating surface of the implant such that when fully seated the terminal surface of said fixation members is flush or congruent with the external articulating surface of the implant.

13. The implant of claim 9 wherein the implant is formed from a biologically compatible metal, polymer, biologic, allograft, or xenograft material.

14. The implant of claim 9 wherein fixation of the implant to the femur is augmented by bone cement or adhesive that is be placed between the implant and the femur.

15. The implant of claim 9 wherein the implant is of such a size and configuration that it may be placed into the knee joint through an external skin incision that measures 3 cm or less.

16. The implant of claim 9 wherein the implant may be implanted using an arthroscopic visualization method.

17. An implant to resurface a femoral condyle of the knee joint comprising a femoral body that has a distal, convex surface with a profile that closely approximates the radius of curvature of the articulating surface of a native femoral condyle in the sagital plane for articulation with the tibial side of the joint and that has a proximal surface with an apex for engagement with cancellous bone of the distal end of a prepared femur and one or more supports for engagement with cortical bone of the distal end of a prepared femur.

18. The implant of claim 17 wherein fixation of the implant to the femur is provided by one or more screws that are inserted in one or more screw receiving holes that traverse the implant through the femoral body from the distal, convex surface to the proximal surface.

19. The implant of claim 17 wherein fixation is provided by one or more rigid fixation members, such as screws, pegs, pins or stems, that are inserted in one or more screw receiving holes that traverse the implant through the femoral body from the distal, convex surface to the proximal surface and that traverse the external articulating surface and have a terminal surface profile that uniformly matches the distal, convex articulating surface of the implant such that when fully seated the terminal surface of said fixation members is flush or congruent with the external articulating surface of the implant.

20. The implant of claim 17 wherein the implant is formed from a biologically compatible metal, polymer, biologic, allograft, or xenograft material.

21. The implant of claim 17 wherein fixation of the implant to the femur is augmented by bone cement or adhesive that is be placed between the implant and the femur.

22. The implant of claim 17 wherein the implant is of such a size and configuration that it may be placed into the knee joint through an external skin incision that measures 3 cm or less.

23. The implant of claim 17 wherein the implant may be implanted using an arthroscopic visualization method.

24. An implant to resurface a femoral condyle of the knee joint comprising a femoral body that has a distal, convex surface with a profile that closely approximates the radius of curvature of the articulating surface of a native femoral condyle in the sagital plane for articulation with the tibial side of the joint and that has a proximal surface with an apex for engagement with cancellous bone of the distal end of a prepared femur and one or more supports for engagement with cortical bone of the distal end of a prepared femur and where the fixation of the implant to the femur is provided by one or more screws that are inserted in one or more screw receiving holes that traverse the implant through the femoral body from the distal, convex surface to the proximal surface and the implant is formed from a biologically compatible metal, polymer, biologic, allograft, or xenograft material.

25. The implant of claim 24 wherein fixation of the implant to the femur is augmented by bone cement or adhesive that is be placed between the implant and the femur.

26. The implant of claim 24 wherein the implant is of such a size and configuration that it may be placed into the knee joint through an external skin incision that measures 3 cm or less.

27. The implant of claim 24 wherein the implant may be implanted using an arthroscopic visualization method.

28. An implant to resurface a femoral condyle of the knee joint comprising a femoral body that has a distal, convex surface with a profile that closely approximates the radius of curvature of the articulating surface of a native femoral condyle in the sagital plane for articulation with the tibial side of the joint and that has a proximal surface with an apex for engagement with cancerous bone of the distal end of a prepared femur and one or more supports for engagement with cortical bone of the distal end of a prepared femur wherein fixation of the implant is provided by one or more rigid fixation members, such as screws, pegs, pins or stems, that are inserted in one or more screw receiving holes that traverse the implant through the femoral body from the distal, convex surface to the proximal surface and that traverse the external articulating surface and have a terminal surface profile that uniformly matches the distal, convex articulating surface of the implant such that when fully seated the terminal surface of said fixation members is flush or congruent with the external articulating surface of the implant and the implant is formed from a biologically compatible metal, polymer, biologic, allograft, or xenograft material.

29. The implant of claim 28 wherein fixation of the implant to the femur is augmented by bone cement or adhesive placed between the implant and the femur.

30. The implant of claim 28 wherein the implant is of such a size and configuration that it may be placed into the knee joint through an external skin incision that measures 3 cm or less.

31. The implant of claim 28 wherein the implant may be implanted using an arthroscopic visualization method.

32. An implant to resurface a femoral condyle of the knee joint comprising a femoral body that has a distal, convex surface with a profile that closely approximates the radius of curvature of the articulating surface of a native femoral condyle in the sagital plane for articulation with the tibial side of the joint and that has a proximal surface with an apex for engagement with cancellous bone of the distal end of a prepared femur and one or more supports for engagement with cortical bone of the distal end of a prepared femur and where the fixation of the implant to the femur is provided by one or more screws that are inserted in one or more screw receiving holes that traverse the implant through the femoral body from the distal, convex surface to the proximal surface and the implant is formed from a biologically compatible metal, polymer, biologic, allograft, or xenograft material and wherein the implant is of such a size and configuration that it may be placed into the knee joint through an external skin incision that measures 3 cm or less and the implant may be implanted using an arthroscopic visualization method

33. An implant to resurface a femoral condyle of the knee joint comprising a femoral body that has a distal, convex surface with a profile that closely approximates the radius of curvature of the articulating surface of a native femoral condyle in the sagital plane for articulation with the tibial side of the joint and that has a proximal surface with an apex for engagement with cancellous bone of the distal end of a prepared femur and one or more supports for engagement with cortical bone of the distal end of a prepared femur wherein fixation of the implant is provided by one or more rigid fixation members, such as screws, pegs, pins or stems, that are inserted in one or more screw receiving holes that traverse the implant through the femoral body from the distal, convex surface to the proximal surface and that traverse the external articulating surface and have a terminal surface profile that uniformly the distal convex articulating surface of the implant such that when fully seated the terminal surface of said fixation members is flush or congruent with the external articulating surface of the implant and the implant is formed from a biologically compatible metal, polymer, biologic, allograft, or xenograft material and wherein the implant is of such a size and configuration that it may be placed into the knee joint through an external skin incision that measures 3 cm or less and the implant may be implanted using an arthroscopic visualization method.

34. An implant to resurface the tibial condyle of the knee joint comprising a tibial body that has a concave superior surface, a planar inferior surface, and divided into medial and lateral halves by a joint running longitudinally through the implant.

35. The implant of claim 34 wherein the medial and lateral halves of the tibial body may be passed separately into the joint space of a knee joint and be assembled into a single unit therein.

36. The implant of claim 34 wherein the medial and lateral halves are self-locking or include a separate locking device to maintain their attachment within the knee joint following assembly into a single unit.

37. The implant of claim 36 wherein the medial and lateral halves of the tibial body include complementary dovetail joints or other geometric interface junctions to permit assembly of the halves into a single unit.

38. The implant of claim 34 wherein the tibial component is free floating upon the tibial surface.

39. The implant of claim 35 wherein the tibial component is securely attached to the tibia.

40. The implant of claim 34 wherein the tibial component is of such a size and configuration that it may be placed into the knee joint through an external skin incision that measures 3 cm or less.

41. The implant of claim 34 wherein the tibial implant component may be attached to the tibia by means placed through an oblique access channel formed through the tibia from the anterior tibial cortex to the central tibial condylar surface.

42. The implant of claim 41 wherein the tibial implant component is secured to the tibia by a cement, adhesive, or biologically active substance applied to the inferior tibial implant component surface through the oblique access channel.

43. The implant of claim of 41 wherein the tibial implant component is secured to the tibia by a strand or member that is attached to the inferior tibial implant component surface and which passes into the oblique access channel.

44 The implant of claim 41 wherein said strand or member extends through the length of the oblique access channel and exits through the opening of the oblique access channel on the anterior tibial cortex.

45. The implant of claim 44 wherein the strand or member is externally attached to the anterior tibial cortex to secure the tibial implant component to the tibial surface.

46. The implant of claim 45 wherein the strand or member is under tension when said strand or member is attached to the anterior tibial cortex to secure the tibial implant component to the tibial surface.

47. The implant of claim 41 wherein the strand or member is a suture, cable, chain, screw, rod, bolt, loop, hook, or grasping device.

48. An implant to resurface the tibial condyle of the knee joint comprising a tibial body that has a semicircular circumference, a concave superior surface, a planar inferior surface, and divided into medial and lateral halves by a joint running longitudinally through the implant.

49. The implant of claim 48 wherein the medial and lateral halves of the tibial body may be passed separately into the joint space of a knee joint and be assembled into a single unit therein.

50. The implant of claim 48 wherein the medial and lateral halves are self-locking or include a separate locking device to maintain their attachment following assembly into a single unit.

51. The implant of claim 50 wherein the medial and lateral halves of the tibial body include complementary dovetail joints or other geometric interface junctions to permit assembly of the halves into a single unit.

52. The implant of claim 48 wherein the tibial component is free floating upon the tibial surface.

53. The implant of claim 48 wherein the tibial component is securely attached to the tibia.

54. The implant of claim 48 wherein the tibial component is of such a size and configuration that it may be placed into the knee joint through an external skin incision that measures 3 cm or less.

55. The implant of claim 48 wherein the tibial implant component may be attached to the tibia by means placed through an oblique access channel formed through the tibia from the anterior tibial cortex to the central tibial condylar surface.

56. The implant of claim 55 wherein the tibial implant component is secured to the tibia by a cement, adhesive, or biologically active substance applied to the inferior tibial implant component surface through the oblique access channel.

57. The implant of claim of 55 wherein the tibial implant component is secured to the tibia by a strand or member that is attached to the inferior tibial implant component surface and which passes into the oblique access channel.

58 The implant of claim 57 wherein said strand or member extends through the length of the oblique access channel and exits through the opening of the access channel on the anterior tibial cortex.

59. The implant of claim 58 wherein the strand or member is externally attached to the anterior tibial cortex to secure the tibial implant component to the tibial surface.

60. The implant of claim 59 wherein the strand or member is under tension when said strand or member is attached to the anterior tibial cortex to secure the tibial implant component to the tibial surface.

61. The implant of claim 55 wherein the strand or member is a suture, cable, chain, screw, rod, bolt, loop, hook, or grasping device.

62. An implant to resurface the tibial condyle of the knee joint comprising a tibial body that has a concave superior surface, a planar inferior surface, and divided into medial and lateral halves by a joint running longitudinally through the implant where the medial and lateral halves of the tibial body may be passed separately into the joint space of a knee joint and be assembled into a single unit therein.

63. The implant of claim 62 wherein the medial and lateral halves are self-locking or include a separate locking device to maintain their attachment following assembly into a single unit.

64. The implant of claim 63 wherein the medial and lateral halves of the tibial body include complementary dovetail joints or other geometric interface junctions to permit assembly of the halves into a single unit.

65. The implant of claim 62 wherein the tibial component is free floating upon the tibial surface.

66. The implant of claim 62 wherein the tibial component is securely attached to the tibia.

67. The implant of claim 62 wherein the tibial component is of such a size and configuration that it may be placed into the knee joint through an external skin incision that measures 3 cm or less.

68. The implant of claim 62 wherein the tibial implant component may be attached to the tibia by means placed through an oblique access channel formed through the tibia from the anterior tibial cortex to the central tibial condylar surface.

69. The implant of claim 68 wherein the tibial implant component is secured to the tibia by a cement, adhesive, or biologically active substance applied to the inferior tibial implant component surface through the oblique access channel.

70. The implant of claim of 68 wherein the tibial implant component is secured to the tibia by a strand or member that is attached to the inferior tibial implant component surface and which passes into the oblique access channel.

71. The implant of claim 70 wherein said strand or member extends through the length of the oblique access channel and exits through the opening of the oblique access channel on the anterior tibial cortex.

72. The implant of claim 71 wherein the strand or member is externally attached to the anterior tibial cortex to secure the tibial implant component to the tibial surface.

73. The implant of claim 72 wherein the strand or member is under tension when said strand or member is attached to the anterior tibial cortex to secure the tibial implant component to the tibial surface.

74. The implant of claim 70 wherein the strand or member is a suture, cable, chain, screw, rod, bolt, loop, hook, or grasping device.

75. An implant to resurface the tibial condyle of the knee joint comprising a tibial body that has a semicircular circumference, a concave superior surface, a planar inferior surface, and divided into medial and lateral halves by a joint running longitudinally through the implant where the medial and lateral halves of the tibial body may be passed separately into the joint space of a knee joint and be assembled into a single unit therein.

76. The implant of claim 75 wherein the medial and lateral halves are self-locking or include a separate locking device to maintain their attachment following assembly into a single unit.

77. The implant of claim 76 wherein the medial and lateral halves of the tibial body include complementary dovetail joints or other geometric interface junctions to permit assembly of the halves into a single unit.

76. The implant of claim 75 wherein the tibial component is free floating upon the tibial surface.

77. The implant of claim 75 wherein the tibial component is securely attached to the tibia.

78. The implant of claim 75 wherein the tibial component is of such a size and configuration that it may be placed into the knee joint through an external skin incision that measures 3 cm or less.

79. The implant of claim 75 wherein the tibial implant component may be attached to the tibia by means placed through an oblique access channel formed through the tibia from the anterior tibial cortex to the central tibial condylar surface.

80. The implant of claim 79 wherein the tibial implant component is secured to the tibia by a cement, adhesive, or biologically active substance applied to the inferior tibial implant component surface through the oblique access channel.

81. The implant of claim of 79 wherein the tibial implant component is secured to the tibia by a strand or member that is attached to the inferior tibial implant component surface and which passes into the oblique access channel.

82. The implant of claim 81 wherein said strand or member extends through the length of the oblique access channel and exits through the opening of the oblique access channel on the anterior tibial cortex.

83. The implant of claim 82 wherein the strand or member is externally attached to the anterior tibial cortex to secure the tibial implant component to the tibial surface.

84. The implant of claim 83 wherein the strand or member is under tension when said strand or member is attached to the anterior tibial cortex to secure the tibial implant component to the tibial surface.

85. The implant of claim 81 wherein the strand or member is a suture, cable, chain, screw, rod, bolt, loop, hook, or grasping device.

86. A knee replacement implant comprising:
a femoral implant component to resurface a femoral condyle of the knee joint that has a curved convex surface for articulation with the tibial side of the joint and the opposite surface composed of an apex for engagement with a prepared femur; and
a tibial implant component implant to resurface the tibial condyle of the knee joint that has a semicircular circumference, a concave superior surface, a planar inferior surface, and divided into medial and lateral halves by a joint running longitudinally through the implant.

87. The knee replacement implant of claim 86 wherein the opposite surface of the femoral implant component also includes one or more supports.

88. The knee replacement implant of claim 86 wherein the medial and lateral halves of the tibial implant component may be passed separately into the joint space of a knee joint and there be assembled into a single unit.

89. The knee replacement implant of claim 86 wherein the opposite surface of the femoral implant component also includes one or more supports and the medial and lateral halves of the tibial implant component may be passed separately into the joint space of a knee joint and there be assembled into a single unit.

90. The knee replacement implant of claim 89 wherein the size and configuration of the femoral implant component and of the medial and lateral halves of the tibial implant component are such that they may be placed into the knee joint through an external skin incision that measures 3 cm or less.

91. The knee replacement implant of claim 86 wherein the tibial implant component may be attached to the tibia by means placed through an oblique access channel formed through the tibia from the anterior tibial cortex to the central tibial condylar surface.

92. The implant of claim 91 wherein the tibial implant component is secured to the tibia by a cement, adhesive, or biologically active substance applied to the inferior tibial implant component surface through the oblique access channel.

93. The implant of claim of 91 wherein the tibial implant component is secured to the tibia by a strand or member that is attached to the inferior tibial implant component surface and which passes into the oblique access channel.

94. The knee replacement implant of claim 91 wherein the size and configuration of the femoral implant component and of the medial and lateral halves of the tibial implant component are such that they may be placed into the knee joint through an external skin incision that measures 3 cm or less.
